(19) United States
(12) Patent Application Publication
(10) Pub. No.: US 2015/0222139 A1
(43) Pub. Date: Aug. 6, 2015

(54) WIRELESS BATTERY CHARGING

(71) Applicant: THORATEC CORPORATION, Pleasanton, CA (US)

(72) Inventors: Ethan Petersen, Oakland, CA (US); John Freddy Hansen, Pleasanton, CA (US)

(21) Appl. No.: 14/414,708

(22) PCT Filed: Jul. 29, 2013
(86) PCT No.: PCT/US2013/052522

§ 371 Date: Jan. 14, 2015

Related U.S. Application Data
(60) Provisional application No. 61/676,629, filed on Jul. 27, 2012.

(51) Int. Cl. H02J 7/02; A61M 1/12; H02J 5/00

(52) U.S. Cl. CPC H02J 7/025; H02J 5/005; A61M 1/127

(57) ABSTRACT

Systems and designs for charging a battery in a wireless power transfer system are provided, which may include any number of features. In one embodiment, an implantable wireless power receiver is coupled to a battery with a rectifier circuit. The receiver can include battery monitor circuitry configured to measure a parameter of the battery. The receiver can be configured to wirelessly communicate the parameter of the battery to the transmitter, which can control delivery of power from the transmitter to the receiver based on the measured battery parameter. Power can be delivered to the battery without utilizing a battery charge control circuit in the device or receiver. Methods of use are also provided.

### CROSS-REFERENCE TO RELATED APPLICATIONS

This application claims the benefit of U.S. Provisional Patent Application No. 61/676,629, filed Jul. 27, 2012, titled “Wireless Battery Charging”.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates generally to methods and apparatus for transmitting and receiving power wirelessly, and in various respects, mechanical circulatory support.

### BACKGROUND

Powered devices need to have a mechanism to supply power to the operative parts. Typically systems use a physical power cable to transfer energy over a distance. There has been a continuing need for systems that can transmit power efficiently over a distance without physical structures bridging the physical gap.

Systems and methods that supply power without electrical wiring are sometimes referred to as wireless energy transmission (WET). Wireless energy transmission greatly expands the types of applications for electrically powered devices. One such example is the field of implantable medical devices. Implantable medical devices typically require an internal power source able to supply adequate power for the reasonable lifetime of the device or an electrical cable that traverses the skin. Typically an internal power source (e.g., a battery) is feasible for only low power devices like sensors. Likewise, a transcutaneous power cable significantly affects patient quality of life (QoL), infection risk, and product life, among many other drawbacks.

More recently there has been an emphasis on systems that supply power to an implanted device without using transcutaneous wiring. Such a system is sometimes referred to as a Transcutaneous Energy Transfer (TET) system. Frequently energy transfer is accomplished using two magnetically coupled coils set up like a transformer to transfer power magnetically across the skin. Conventional TET systems are relatively sensitive to variations in position and alignment of the coils. In order to provide constant and adequate power, the two coils need to be physically close together and well aligned.

TET systems can be combined with implantable medical devices in order to reliably power those devices. In these examples, the TET system recharges the implanted battery of the medical device. A TET system can transfer power to an implanted device from an external power source. The power can then used to run the device and recharge the implanted battery. A typical system would have the TET system output connected to a regulator to control the voltage on a DC bus. There would then be a battery charger that regulates power from the DC bus to the battery.

### SUMMARY OF THE DISCLOSURE

A wireless power transfer system is provided, comprising a device comprising a battery and a load, a receiver adapted to receive wireless power and deliver received power to the battery, the receiver having an output coupled via a rectifier to the device, the receiver including electronics configured to measure a parameter of the battery and communicate the measured parameter wirelessly, and a transmitter inductively coupled to the receiver, the transmitter being driven by a power source and a transmit controller, wherein the transmit controller is configured to control delivery of wireless power from the transmitter to the receiver based on the measured battery parameter.

In some embodiments, the device is a medical device, the device and the receiver being adapted to be implanted in a patient, and the power transmitter is adapted to transmit power to the receiver from a position external to the patient.

In one embodiment, the receiver is configured to deliver received power to the battery of the device without utilizing a battery charge control circuit in the device or receiver.

In another embodiment, the electronics comprise a battery monitor circuit configured to measure a voltage of the battery. In one embodiment, the electronics comprise a battery monitor circuit configured to measure a current of the battery. In some embodiments, the electronics comprise a battery monitor circuit configured to measure a temperature of the battery. In one embodiment, the electronics comprise a battery monitor circuit configured to measure a state of charge of the battery.

In some embodiments, a receive controller of the receiver is configured to communicate the measured parameter wirelessly to the transmitter. In another embodiment, the receive controller is configured to communicate the measured parameter via a radio communications channel. In one embodiment, the receive controller is configured to communicate the measured parameter modulation of the received wireless power.

A method of charging a battery with a wireless power transfer system having an inductively coupled transmitter and receiver is also provided, comprising the steps of measuring a battery parameter of a device coupled to the receiver, wirelessly communicating the battery parameter from the receiver to the transmitter, delivering wireless power from the transmitter to the receiver based on the battery parameter without utilizing a battery charge control circuit in the device or receiver, and delivering power received by the receiver to the battery.

In some embodiments, the battery and the receiver are implanted in a patient and the transmitter is external to the patient.

In another embodiment, the wirelessly communicating step further comprises wirelessly communicating the battery parameter with a radio communications link between the receiver and the transmitter.

In some embodiments, the wirelessly communicating step further comprises wirelessly communicating the battery parameter with modulation between the receiver and the transmitter.

In some embodiments, the battery parameter comprises a voltage of the battery, a current of the battery, a temperature of the battery, or a state of charge of the battery.

A wireless power transfer system is provided, comprising an external wireless power transmitter comprising a transmitter coil, an implantable wireless power receiver comprising a receiver coil inductively coupled to the transmitter coil of the wireless power transmitter, the wireless power receiver comprising a rectifier on an output of the wireless power receiver, an implantable medical device comprising a battery and a load, the implantable medical device being coupled to the rectifier of the wireless power receiver, a battery measurement circuit disposed in the wireless power receiver and being configured to measure a parameter of the battery; a receive controller disposed in the receiver and being configured to communicate the measured parameter wirelessly to the wireless power transmitter, and a transmit controller disposed in the wireless power transmitter and being configured to control delivery of power from the wireless power transmitter to the wireless power receiver based on the measured battery parameter received from the receiver.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates a basic wireless power transfer system.

FIG. 2 illustrates the flux generated by a pair of coils.

FIGS. 3A-3B illustrate the effect of coil alignment on the coupling coefficient.

FIG. 4 illustrates a TET system coupled to a separate implantable medical device.

### DETAILED DESCRIPTION

In the description that follows, like components have been given the same reference numerals, regardless of whether they are shown in different embodiments. To illustrate an embodiment(s) of the present disclosure in a clear and concise manner, the drawings may not necessarily be to scale and certain features may be shown in somewhat schematic form. Features that are described and/or illustrated with respect to one embodiment may be used in the same way or in a similar way in one or more other embodiments and/or in combination with or instead of the features of the other embodiments.

Various aspects of the invention are similar to those described in International Patent Pub. No. WO2012045050; U.S. Pat. Nos. 8,140,168; 7,865,245; 7,774,069; 7,711,433; 7,650,187; 7,571,007; 7,741,734; 7,825,543; 6,591,139; 6,553,263; and 5,350,413; and U.S. Pub. Nos. 2010/0308939; 2008/027293; and 2010/0102639, the entire contents of which patents and applications are incorporated herein for all purposes.

### Wireless Power Transmission System

Power may be transmitted wirelessly by magnetic induction. In various embodiments, the transmitter and receiver are closely coupled.

In some cases “closely coupled” or “close coupling” refers to a system that requires the coils to be very near each other in order to operate. In some cases “loosely coupled” or “loose coupling” refers to a system configured to operate when the coils have a significant spatial and/or axial separation, and in some cases up to distance equal to or less than the diameter of the larger of the coils. In some cases, “loosely coupled” or “loose coupling” refers a system that is relatively insensitive to changes in physical separation and/or orientation of the receiver and transmitter.

In various embodiments, the transmitter and receiver are non-resonant coils. For example, a change in current in one coil induces a changing magnetic field. The second coil within the magnetic field picks up the magnetic flux, which in turn induces a current in the second coil. An example of a closely coupled system with non-resonant coils is described in International Pub. No. WO2000/074747, incorporated herein for all purposes by reference. A conventional transformer is another example of a closely coupled, non-resonant system. In various embodiments, the transmitter and receiver are resonant coils. For example, one or both of the coils is connected to a tuning capacitor or other means for controlling the frequency in the respective coil. An example of closely coupled system with resonant coils is described in International Pub. Nos. WO2001/037926; WO2012/087807; WO2012/087811; WO2012/087816; WO2012/087819; WO2010/030378; and WO2012/056365, and U.S. Pub. No. 2003/0171792, incorporated herein for all purposes by reference.

In various embodiments, the transmitter and receiver are loosely coupled. For example, the transmitter can resonate to propagate magnetic flux that is picked up by the receiver at relatively great distances. In some cases energy can be transmitted over several meters. In a loosely coupled system power transfer may not necessarily depend on a critical distance. Rather, the system may be able to accommodate changes to the coupling coefficient between the transmitter and receiver. An example of a loosely coupled system is described in International Pub. No. WO2012/045050, incorporated herein for all purposes by reference.

Power may be transmitted wirelessly by radiating energy. In various embodiments, the system comprises antennas. The antennas may be resonant or non-resonant. For example, non-resonant antennas may radiate electromagnetic waves to create a field. The field can be near field or far field. The field can be directional. Generally far field has greater range but a lower power transfer rate. An example of such a system for radiating energy with resonators is described in International Pub. No. WO2010/089354, incorporated herein for all purposes by reference. An example of such a non-resonant system is described in International Pub. No. WO2009/018271, incorporated herein for all purposes by reference. Instead of antenna, the system may comprise a high energy light source such as a laser. The system can be configured so photons carry electromagnetic energy in a spatially restricted, direct, coherent path from a transmission point to a receiving point. An example of such a system is described in International Pub. No. WO2010/089354, incorporated herein for all purposes by reference.

Power may also be transmitted by taking advantage of the material or medium through which the energy passes. For example, volume conduction involves transmitting electrical energy through tissue between a transmitting point and a receiving point. An example of such a system is described in International Pub. No. WO2008/066941, incorporated herein for all purposes by reference.

Power may also be transferred using a capacitor charging technique. The system can be resonant or non-resonant. Exemplars of capacitor charging for wireless energy transfer are described in International Pub. No. WO2012/056365, incorporated herein for all purposes by reference.

The system in accordance with various aspects of the invention will now be described in connection with a system for wireless energy transfer by magnetic induction. The exemplary system utilizes resonant power transfer. The system works by transmitting power between the two inductively coupled coils. In contrast to a transformer, however, the exemplary coils are not coupled together closely. A transformer generally requires the coils to be aligned and positioned directly adjacent each other. The exemplary system accommodates looser coupling of the coils.

While described in terms of one receiver coil and one transmitter coil, one will appreciate from the description herein that the system may use two or more receiver coils and two or more transmitter coils. For example, the transmitter may be configured with two coils—a first coil to resonate flux and a second coil to excite the first coil. One will further appreciate from the description herein that usage of “resonator” and “coil” may be used somewhat interchangeably. In various respects, “resonator” refers to a coil and a capacitor connected together.

In accordance with various embodiments of this disclosure, the system comprises one or more transmitters configured to transmit power wirelessly to one or more receivers. In various embodiments, the system includes a transmitter and more than one receiver in a multiplexed arrangement. A frequency generator may be electrically coupled to the transmitter to drive the transmitter to transmit power at a particular frequency or range of frequencies. The frequency generator can include a voltage controlled oscillator and one or more switchable arrays of capacitors, a voltage controlled oscillator and one or more varactors, a phase-locked-loop, a direct digital synthesizer, or combinations thereof. The transmitter can be configured to transmit power at multiple frequencies simultaneously. The frequency generator can include two or more phase-locked-loops electrically coupled to a common reference oscillator, two or more independent voltage controlled oscillators, or combinations thereof. The transmitter can be arranged to simultaneously delivery power to multiple receivers at a common frequency.

In various embodiments, the transmitter is configured to transmit a low power signal at a particular frequency. The transmitter may transmit the low power signal for a particular time and/or interval. In various embodiments, the transmitter is configured to transmit a high power signal wirelessly at a particular frequency. The transmitter may transmit the high power signal for a particular time and/or interval.

In various embodiments, the receiver includes a frequency selection mechanism electrically coupled to the receiver coil and arranged to allow the resonator to change a frequency or a range of frequencies that the receiver can receive. The frequency selection mechanism can include a switchable array of discrete capacitors, a variable capacitance, one or more inductors electrically coupled to the receiving antenna, additional turns of a coil of the receiving antenna, or combinations thereof.

In general, most of the flux from the transmitter coil does not reach the receiver coil. The amount of flux generated by the transmitter coil that reaches the receiver coil is described by “k” and referred to as the “coupling coefficient.”

In various embodiments, the system is configured to maintain a value of k in the range of between about 0.2 to about 0.01. In various embodiments, the system is configured to maintain a value of k of at least 0.01, at least 0.02, at least 0.03, at least 0.04, at least 0.05, at least 0.1, or at least 0.15.

In various embodiments, the coils are physically separated. In various embodiments, the separation is greater than a thickness of the receiver coil. In various embodiments, the separation distance is equal to or less than the diameter of the larger of the receiver and transmitter coil.

Because most of the flux does not reach the receiver, the transmitter coil must generate a much larger field than what is coupled to the receiver. In various embodiments, this is accomplished by configuring the transmitter with a large number of amp-turns in the coil.

Since only the flux coupled to the receiver gets coupled to a real load, most of the energy in the field is reactive. The current in the coil can be sustained with a capacitor connected to the coil to create a resonator. The power source thus only needs to supply the energy absorbed by the receiver. The resonant capacitor maintains the excess flux that is not coupled to the receiver.

In various embodiments, the impedance of the receiver is matched to the transmitter. This allows efficient transfer of energy out of the receiver. In this case the receiver coil may not need to have a resonant capacitor.

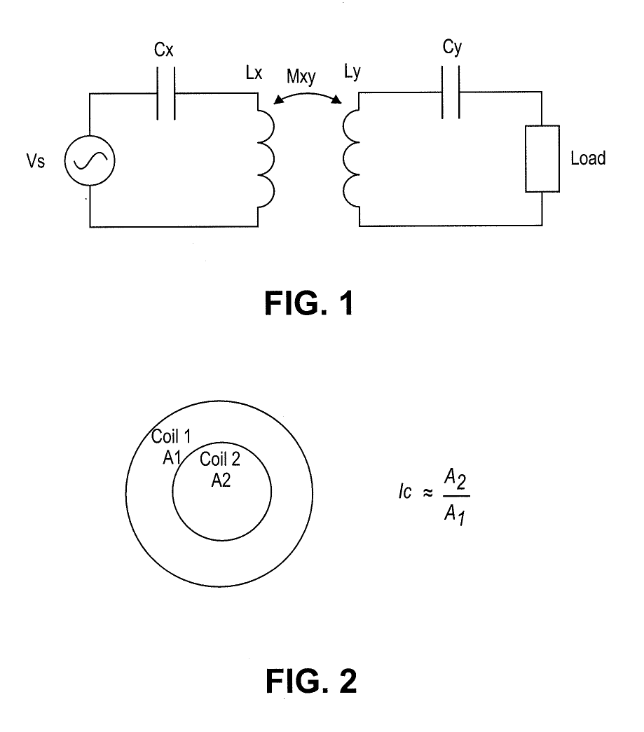

Turning now to FIG. 1, a simplified circuit for wireless energy transmission is shown. The exemplary system shows a series connection, but the system can be connected as either series or parallel on either the transmitter or receiver side.

The exemplary transmitter includes a coil Lx connected to a power source Vs by a capacitor Cx. The exemplary receiver includes a coil Ly connected to a load by a capacitor Cy. Capacitor Cx may be configured to make Lx resonate at a desired frequency. Capacitance Cx of the transmitter coil may be defined by its geometry. Inductors Lx and Ly are connected by coupling coefficient k. Mxy is the mutual inductance between the two coils. The mutual inductance, Mxy, is related to coupling coefficient, k.

Mxy=k\sqrt{Lx·Ly}

In the exemplary system the power source Vs is in series with the transmitter coil Lx so it may have to carry all the reactive current. This puts a larger burden on the current rating of the power source and any resistance in the source will add to losses.

The exemplary system includes a receiver configured to receive energy wirelessly transmitted by the transmitter. The exemplary receiver is connected to a load. The receiver and load may be connected electrically with a controllable switch.

In various embodiments, the receiver includes a circuit element configured to be connected or disconnected from the receiver coil by an electronically controllable switch. The electrical coupling can include both a serial and parallel arrangement. The circuit element can include a resistor, capacitor, inductor, lengths of an antenna structure, or combinations thereof. The system can be configured such that power is transmitted by the transmitter and can be received by the receiver in predetermined time increments.

In various embodiments, the transmitter coil and/or the receiver coil is a substantially two-dimensional structure. In various embodiments, the transmitter coil may be coupled to a transmitter impedance-matching structure. Similarly, the receiver coil may be coupled to a receiver impedance-matching structure. Examples of suitable impedance-matching structures include, but are not limited to, a coil, a loop, a transformer, and/or any impedance-matching network. An impedance-matching network may include inductors or capacitors configured to connect a signal source to the resonator structure.

In various embodiments, the transmitter is controlled by a controller (not shown) and driving circuit. The controller and/or driving circuit may include a directional coupler, a signal generator, and/or an amplifier. The controller may be configured to adjust the transmitter frequency or amplifier gain to compensate for changes to the coupling between the receiver and transmitter.

In various embodiments, the transmitter coil is connected to an impedance-matched coil loop. The loop is connected to a power source and is configured to excite the transmitter coil. The first coil loop may have finite output impedance. A signal generator output may be amplified and fed to the transmitter coil. In use power is transferred magnetically between the first coil loop and the main transmitter coil, which in turns transmits flux to the receiver. Energy received by the receiver coil is delivered by Ohmic connection to the load.

One of the challenges to a practical circuit is how to get energy in and out of the resonators. Simply putting the power source and load in series or parallel with the resonators is difficult because of the voltage and current required. In various embodiments, the system is configured to achieve an approximate energy balance by analyzing the system characteristics, estimating voltages and currents involved, and controlling circuit elements to deliver the power needed by the receiver.

In an exemplary embodiment, the system load power, P_{L}, is assumed to be 15 Watts and the operating frequency, f, is 250 kHz. Then, for each cycle the load removes a certain amount of energy from the resonance:

$${e}_{L} = \frac{{P}_{L}}{f} = 60 μ J$$

Energy the load removes in one cycle

It has been found that the energy in the receiver resonance is typically several times larger than the energy removed by the load for operative, implantable medical devices. In various embodiments, the system assumes a ratio 7:1 for energy at the receiver versus the load removed. Under this assumption, the instantaneous energy in the exemplary receiver resonance is 420 μJ.

The exemplary circuit was analyzed and the self inductance of the receiver coil was found to be 60 uH. From the energy and the inductance, the voltage and current in the resonator could be calculated.

$${e}_{y} = \frac{1}{2} {Li}^{2}$$

$${i}_{y} = \sqrt{\frac{2 {e}_{y}}{L}} = 3.74 A peak$$

$${v}_{y} = ω {L}_{y} {i}_{y} = 352 V peak$$

The voltage and current can be traded off against each other. The inductor may couple the same amount of flux regardless of the number of turns. The Amp-turns of the coil needs to stay the same in this example, so more turns means the current is reduced. The coil voltage, however, will need to increase. Likewise, the voltage can be reduced at the expense of a higher current. The transmitter coil needs to have much more flux. The transmitter flux is related to the receiver flux by the coupling coefficient. Accordingly, the energy in the field from the transmitter coil is scaled by k.

$${e}_{x} = \frac{{e}_{y}}{k}$$

Given that k is 0.05:

$${e}_{x} = \frac{420 μ J}{0.05} = 8.4 mJ$$

For the same circuit the self inductance of the transmitter coil was 146 uH as mentioned above. This results in:

$${i}_{x} = \sqrt{\frac{2 {e}_{x}}{L}} = 10.7 A peak$$

$${v}_{x} = ω {L}_{x} {i}_{x} = 2460 V peak$$

One can appreciate from this example, the competing factors and how to balance voltage, current, and inductance to suit the circumstance and achieve the desired outcome. Like the receiver, the voltage and current can be traded off against each other. In this example, the voltages and currents in the system are relatively high. One can adjust the tuning to lower the voltage and/or current at the receiver if the load is lower.

### Estimation of Coupling Coefficient and Mutual Inductance

As explained above, the coupling coefficient, k, may be useful for a number of reasons. In one example, the coupling coefficient can be used to understand the arrangement of the coils relative to each other so tuning adjustments can be made to ensure adequate performance. If the receiver coil moves away from the transmitter coil, the mutual inductance will decrease, and ceteris paribus, less power will be transferred. In various embodiments, the system is configured to make tuning adjustments to compensate for the drop in coupling efficiency.

The exemplary system described above often has imperfect information. For various reasons as would be understood by one of skill in the art, the system does not collect data for all parameters. Moreover, because of the physical gap between coils and without an external means of communications between the two resonators, the transmitter may have information that the receiver does not have and vice versa. These limitations make it difficult to directly measure and derive the coupling coefficient, k, in real time.

Described below are several principles for estimating the coupling coefficient, k, for two coils of a given geometry. The approaches may make use of techniques such as Biot-Savart calculations or finite element methods. Certain assumptions and generalizations, based on how the coils interact in specific orientations, are made for the sake of simplicity of understanding. From an electric circuit point of view, all the physical geometry permutations can generally lead to the coupling coefficient.

If two coils are arranged so they are in the same plane, with one coil circumscribing the other, then the coupling coefficient can be estimated to be roughly proportional to the ratio of the area of the two coils. This assumes the flux generated by coil 1 is roughly uniform over the area it encloses as shown in FIG. 2.

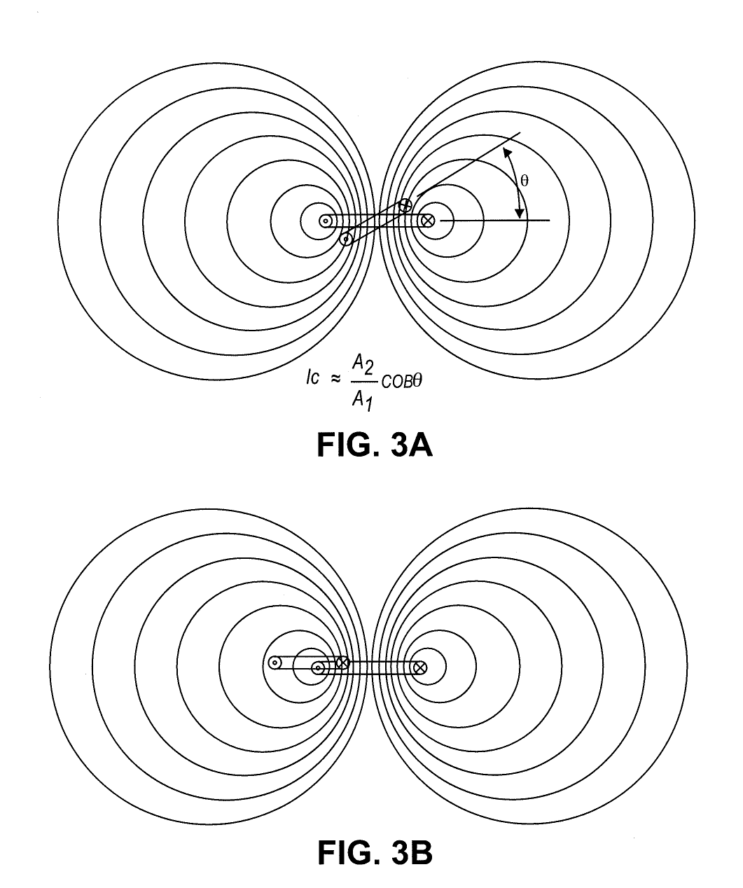

If the coils are out of alignment such that the coils are at a relative angle, the coupling coefficient will decrease. The amount of the decrease is estimated to be about equal to the cosine of the angle as shown in FIG. 3A. If the coils are orthogonal to each other such that theta (0) is 90 degrees, the flux will not be received by the receiver and the coupling coefficient will be zero.

If the coils are arraigned such that half the flux from one coil is in one direction and the other half is in the other direction, the flux cancels out and the coupling coefficient is zero, as shown in FIG. 3B.

A final principle relies on symmetry of the coils. The coupling coefficient and mutual inductance from one coil to the other is assumed to be the same regardless of which coil is being energized.

M_{xy}=M_{yx}

As described above, a typical TET system can be subdivided into two parts, the transmitter and the receiver. Control and tuning may or may not operate on the two parts independently. Typically, wireless power transfer systems utilize a battery charging circuit to manage battery charging and to regulate the power delivered into an implanted battery from the power transfer system. This type of system would have the TETS system output connected to a regulator to control the voltage on a DC bus. There would then be a battery charger that regulates power from the DC bus to the battery.

In contrast, one aspect of the present invention uses the TETS system as the power electronics to regulate the power. This disclosure describes methods and techniques for using a TETS system to manage battery charging of a battery within an implantable medical device without requiring an additional battery charge control circuit in the implanted device.

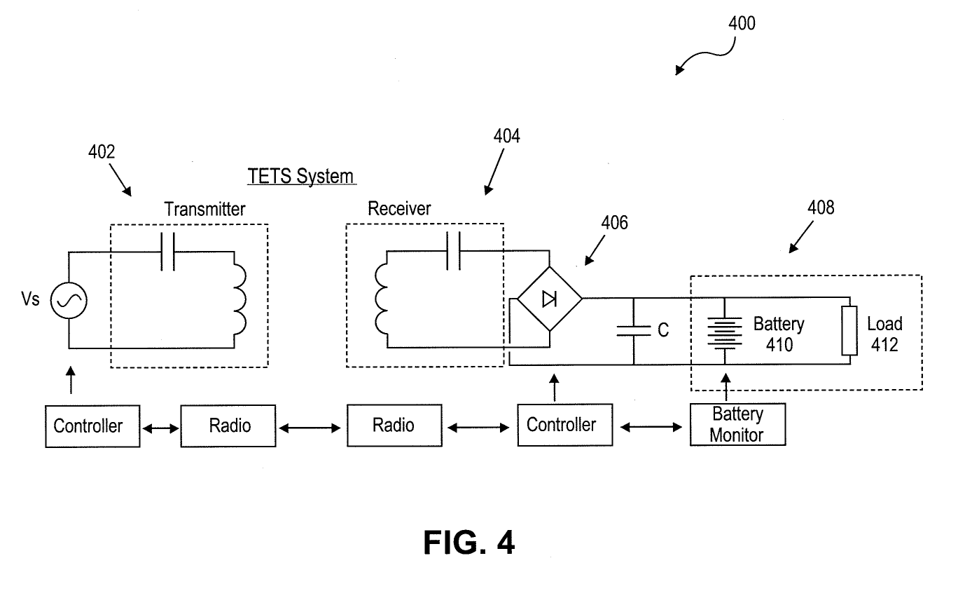

Referring now to FIG. 4, a TET system 400 can have several components, including a transmitter 402 and a receiver 404. The transmitter can be coupled to a power source, Vs, which could be for example, an RF amplifier (e.g., a Class D amplifier) powered by a battery or a power supply plugged into the wall. The receiver can be coupled to a rectifier 406 on the output of the TET system. Additionally, the receiver can be further coupled to a separate implantable medical device 408, which can include a battery 410 and a load 412. In some embodiments, the separate implantable medical device can be implanted in a portion of the patient's body separate from the receiver 404. The load 412 can be any load presented to the receiver 404 by the separate device. The load can comprise, for example, a motor controller running a Left Ventricular Assist Device (LVAD) pump. In FIG. 4, the transmitter can be configured to wirelessly transfer power from outside the body of a patient to the receiver, which can be implanted inside the body of the patient along with the separate implantable medical device.

In this embodiment, the receiver 404 of the TET system can be connected directly to the battery in the implanted device. This embodiment does not include an additional regulator on the implanted side of the system. However, since there is no battery charging circuit or regulator included in this embodiment, this configuration requires some form of communication from the implanted electronics of the receiver to the transmitter to help the transmitter determine how much energy to put into the battery 410.

Although the TET system is able to control how much power is flowing from the transmitter to the receiver, it is desirable to have a way of communicating with the implanted system in order to know the state of the battery in the medical device. There are a couple possibilities for the communications channel. One possibility is in-band communications, where the TETS power signal itself is modulated in a way to communicate information. Alternatively a separate radio can be used, this is sometimes call out of band communications. Other possibilities exist such as optical or acoustic methods. Typically, a TETS receiver would have a communications channel for other purposes, for instance one embodiment uses a radio to communicate other system information, not related to the TETS system. The present embodiment can use the existing communications channel to communicate information for battery charging that does not add any additional pieces to the system.

In one embodiment, a battery monitor circuit in the receiver 404 can detect information about the battery 410 in the implanted medical device 408, such as voltage, current, temperature, battery state of charge, etc, and use the detected information to calculate a parameter of the battery, such as state of charge. This information can be communicated from the battery monitor to a controller of the receiver, which can then communicate that information along the communications channel (e.g., radio) to the controller of transmitter 402. Batteries charge relatively slowly, so latency in the communications channel is not an issue. A delay of a couple seconds will have no significant effect on the charge algorithm.

In various embodiments, the power used to charge the implanted battery is regulated by the TETS transmitter 402. The battery state is monitored by the electronics at the implanted receiver. In one embodiment the implanted receiver electronics can include a battery management IC that can calculate the battery state of change, and a microcontroller to handle the communications. The battery state information can be communicated to the external transmitter over a wireless communication link. The transmitter can then use this information to determine how much power to deliver. For example, the transmitter could then use the battery state information to determine if power needs to be transmitted from the transmitter to the receiver to charge the battery, provide power to the load, or a combination thereof

The system of this disclosure eliminates the regulator and/or battery charging circuit on the output of the TET system (i.e., on the receiver). This simplifies the power electronics in the implanted device. Using the communications between the implanted device and the external power system to communicate battery status allows this simplification of the TET system.

Although described in terms of powering an implanted medical device and charging an implanted battery, this invention can be used for many different wireless power transmission systems. Frequently systems will be powered from a battery and use the wireless power transfer system to recharge the battery. For instance, the invention can be applied to other fields including, but not limited to, automotive electric car charging on a large scale or cell phone charging on a small scale. Moreover, one will appreciate from the description herein that the principles described above can be applied equally using firmware, software, and the like.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A wireless power transfer system, comprising:
a device comprising a battery and a load;
a receiver adapted to receive wireless power and deliver received power to the battery, the receiver having an output coupled via a rectifier to the device, the receiver including electronics configured to measure a parameter of the battery and communicate the measured parameter wirelessly; and
a transmitter inductively coupled to the receiver, the transmitter being driven by a power source and a transmit controller, wherein the transmit controller is configured to control delivery of wireless power from the transmitter to the receiver based on the measured battery parameter.

2. The system of claim 1 wherein the device is a medical device, the device and the receiver being adapted to be implanted in a patient, and the power transmitter is adapted to transmit power to the receiver from a position external to the patient.

3. The system of claim 1 wherein the receiver is configured to deliver received power to the battery of the device without utilizing a battery charge control circuit in the device or receiver.

4. The wireless power transfer system of claim 1, wherein the electronics comprise a battery monitor circuit configured to measure a voltage of the battery.

5. The wireless power transfer system of claim 1, wherein the electronics comprise a battery monitor circuit configured to measure a current of the battery.

6. The wireless power transfer system of claim 1, wherein the electronics comprise a battery monitor circuit configured to measure a temperature of the battery.

7. The wireless power transfer system of claim 1, wherein the electronics comprise a battery monitor circuit configured to measure a state of charge of the battery.

8. The wireless power transfer system of claim 1, wherein a receive controller of the receiver is configured to communicate the measured parameter wirelessly to the transmitter.

9. The wireless power transfer system of claim 8, wherein the receive controller is configured to communicate the measured parameter via a radio communications channel.

10. The wireless power transfer system of claim 8, wherein the receive controller is configured to communicate the measured parameter modulation of the received wireless power.

11. A method of charging a battery with a wireless power transfer system having an inductively coupled transmitter and receiver, comprising the steps of:
measuring a battery parameter of a device coupled to the receiver;
wirelessly communicating the battery parameter from the receiver to the transmitter;
delivering wireless power from the transmitter to the receiver based on the battery parameter without utilizing a battery charge control circuit in the device or receiver; and
delivering power received by the receiver to the battery.

12. The method of claim 11 the battery and the receiver are implanted in a patient and the transmitter is external to the patient.

13. The method of claim 11 wherein the wirelessly communicating step further comprises wirelessly communicating the battery parameter with a radio communications link between the receiver and the transmitter.

14. The method of claim 11 wherein the wirelessly communicating step further comprises wirelessly communicating the battery parameter with modulation between the receiver and the transmitter.

15. The method of claim 11, wherein the battery parameter comprises a voltage of the battery.

16. The method of claim 11, wherein the battery parameter comprises a current of the battery.

17. The method of claim 11, wherein the battery parameter comprises a temperature of the battery.

18. The method of claim 11, wherein the battery parameter comprises a state of charge of the battery.

19. A wireless power transfer system, comprising:
an external wireless power transmitter comprising a transmitter coil;
an implantable wireless power receiver comprising a receiver coil inductively coupled to the transmitter coil of the wireless power transmitter, the wireless power receiver comprising a rectifier on an output of the wireless power receiver;
an implantable medical device comprising a battery and a load, the implantable medical device being coupled to the rectifier of the wireless power receiver;
a battery measurement circuit disposed in the wireless power receiver and being configured to measure battery parameter;
a receive controller disposed in the receiver and being configured to communicate the measured battery parameter wirelessly to the wireless power transmitter; and
a transmit controller disposed in the wireless power transmitter and being configured to control delivery of power from the wireless power transmitter to the wireless power receiver based on the measured battery parameter received from the receiver.
